(19) United States
(12) Patent Application Publication
(10) Pub. No.: US 2007/0023035 A1
(43) Pub. Date: Feb. 1, 2007

(54) Method for processing drugs

(76) Inventors: Kevin M. Kane, Moca, PR (US); Hlrdey Bhathal, San Diego, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/193,096

(22) Filed: Jul. 29, 2005

(51) Int. Cl. A61M 11/00

(52) U.S. Cl. 128/200.14

(57) ABSTRACT

A method of processing a drug is provided that includes dissolving the drug in a solvent to form a drug-containing solution. Droplets of the drug-containing solution are then jetted into a moving volume of gas. The jetted droplets of drug-containing solution have a maximum size of about 1 micron or less.

### BACKGROUND

Frequently drugs from discovery programs or from recently developed research exhibit poor bioavailability due to their low water solubility, low permeability, and other factors. And while the crystalline nature of the drugs can contribute to the poor water solubility, it can also interfere with the combination of such drugs with other materials required to make drug formulations and products.

Reduction of particle size is commonly used to address the low water solubility of drugs because smaller particles exhibit increased dissolution rates. Some methods of processing bulk crystalline drugs for particle size reduction make use of techniques such as milling and blending the crystalline drugs with other materials, such as generally inert substances used as a diluents or vehicles for a drug (known as excipients) to prepare a drug formulation with improved solubility, compressibility for tabletting, and more uniform shape. In some cases, the crystalline habit of the drug particles makes it difficult to obtain a uniform distribution of the drug in the formulation. For example, crystal shape, particle size, and crystalline surface energies of some drugs may interfere in the mixing of the crystalline drug with excipients.

### SUMMARY

A method of processing a drug includes dissolving the drug in a solvent to form a drug-containing solution and jetting droplets of the drug-containing solution into a moving volume of gas. The jetted droplets of drug-containing solution have a maximum size of about 1 micron.

### BRIEF DESCRIPTION OF THE DRAWINGS

The accompanying drawings illustrate various embodiments of the present apparatus and method and are a part of the specification. The illustrated embodiments are merely examples of the present apparatus and method and do not limit the scope of the disclosure.

FIG. 1 illustrates a powder processing system according to one exemplary embodiment.

FIG. 2 is a flowchart illustrating a method of forming a powder according to one exemplary embodiment.

FIG. 3 is a schematic view of a pharmaceutical processing system according to one exemplary embodiment.

Throughout the drawings, identical reference numbers designate similar, but not necessarily identical, elements.

### DETAILED DESCRIPTION

A method and system are provided herein that make use of a material jetting device to produce solid drug particles of relatively small particle size by dispensing small droplets of drug solution into a moving body of gas. The rapid evaporation of droplets that occurs yields a rapidly quenched drug or drug formulation particle in a high-energy, metastable solid state. For example, the resulting solid may be an amorphous particle with a relatively small particle size. Any version of a material jetting device can be used. As used herein, the term droplet shall be broadly understood to mean a volume containing a solvent and a drug or drug formulation. Further, as used herein, the term particle shall be broadly understood to refer to a substasntially solid volume of drug or drug formulation.

According to one exemplary embodiment, the material jetting device includes nozzles that are in communication with a body of drying gas. In particular, according to such an exemplary embodiment, a series of nozzles dispenses laterally into a circular flow of drying gas in a cyclone collector that rapidly dries the droplets to yield solid particles. The circulating flow of gas moves down through a conical collector to separate the particles by centrifugal force into a collection vessel while the particle-lean gas moves out to be exhausted. Dried particles of uniform size, such as a particle size of substantially less than about 1 μm, and approximately spherical in shape are produced. This method may also provide for the continuous monitoring and control of the dispensing conditions and generation of uniformly sized drug particles.

In the following description, for purposes of explanation, numerous specific details are set forth in order to provide a thorough understanding of the present method and apparatus. It will be apparent, however, to one skilled in the art that the present method and apparatus may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

Drug Processing System

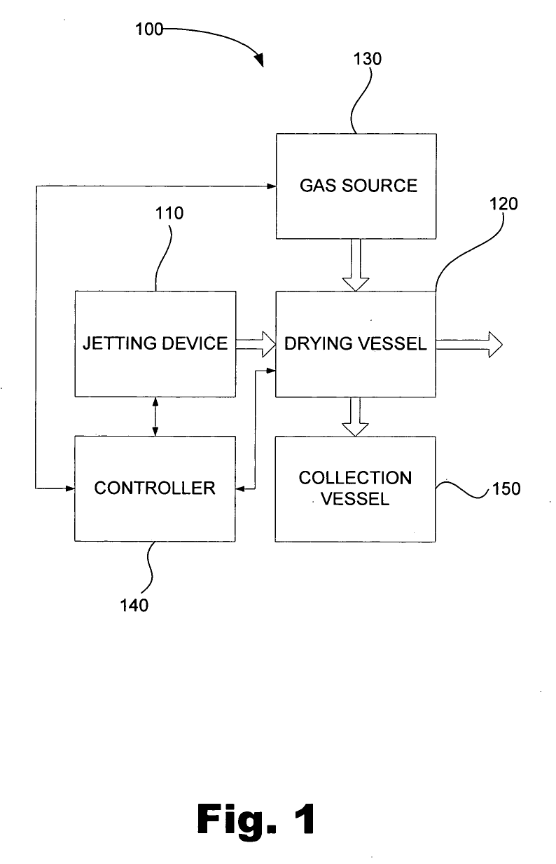

FIG. 1 is a general schematic view of a drug processing system (100) according to one exemplary embodiment. As seen in FIG. 1, the drug processing system (100) generally includes at least one jetting device (110), a drying vessel (120), a gas source (130), a controller (140), and a collection vessel (150). As will be discussed in more detail below, the drug processing system (100) is configured to produce relatively small drug particles that are approximately spherical. In bulk, these particles make up a free-flowing powder. Such particles of a free-flowing powder can be readily processed or dissolved.

As shown in FIG. 1, the gas source (130) is in fluid communication with the drying vessel (120). The gas source (130) establishes and maintains a moving gas volume within the drying vessel (120). For example, according to one exemplary embodiment, the gas source (130) provides a supply of gas to one end of the drying vessel (120). The gas supplied by the gas source (130) then moves through the drying vessel (120) until it is exhausted.

The jetting device (110) is in fluid communication with the drying vessel (120). In particular, according to one exemplary embodiment, the jetting device (110) jets a solution that includes a drug dissolved in a solvent into the drying vessel (120). More specifically, the jetting device (110) jets the drug solution into the drying vessel and into the gas flow established by the gas source (130). The droplets jetted by the jetting device (110) have a relatively small drop size, such as a drop size of less than about 1 μm. The small droplet sizes may allow for relatively rapid drying rates.

A controller (140) is coupled to the jetting device (110), the drying vessel (120), and the gas source (130). The controller (140) monitors the conditions in each of these components to control the resulting particle size. These conditions and their control will be discussed in more detail below.

The gas flow causes the solvent to evaporate into the gas flow, thereby leaving the solid drug. The solid drug is then separated from the gas flow and collected in a collection vessel (150). The solvent-rich gas flow is then exhausted. The resulting solidified drug may have a relatively small particle size and an amorphous structure. The amorphous structure and relatively small particle size may increase the solubility and hence the bioavailability of the drug. One exemplary method of processing such a drug will now be discussed in more detail.

Method of Processing a Drug

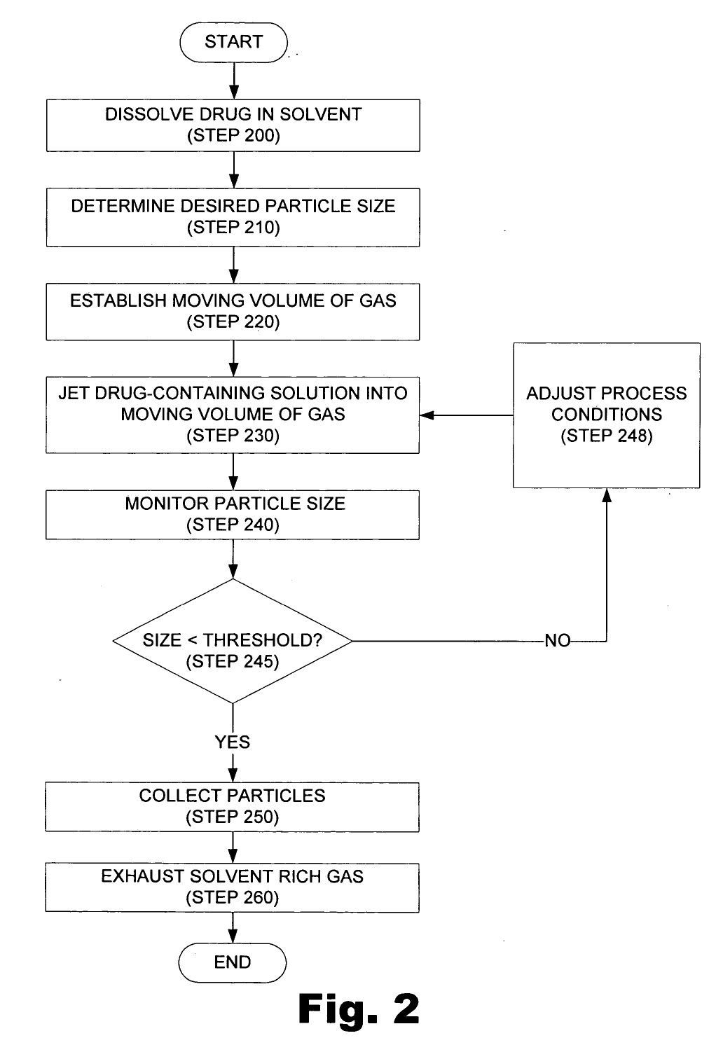

FIG. 2 illustrates an exemplary method of processing a drug. The method begins by dissolving a drug in solvent (step 200). The resulting solution may be referred to as a drug-containing solution. According to one exemplary method, dissolving the drug in a solvent includes dissolving the drug into a readily evaporated liquid. Suitable liquids include, without limitation, low boiling point alcohols, ethers, ketones, esters, halogenated solvents, or the like. In some cases, it may be desirable to include small quantities of water. In addition, combinations of two or more solvents may be advantageous.

The present exemplary method also includes a determination of the desired droplet size (step 210). For example, it may be desirable to produce droplet with a maximum size of less than about 1 micron. Such a droplet size may provide for resulting particles that are also substantially less than about 1 micron in size. Such a particle size may provide for a relatively high surface area per unit volume. A relatively high surface area may increase the relative aqueous solubility of the particles. Further, such a particle size may allow the resulting particles to flow freely as a powder, thereby further increasing the ease of processing the particles in bulk.

A moving volume of gas is then established (step 220). According to one exemplary method, the moving volume of gas is established by directing gas from a gas source to a drying vessel. It may be desirable to control the temperature of the gas used. It may be desirable to use an inert gas in establishing the moving volume of gas. More specifically, the use of an inert gas may reduce the possibility that the gas will undesirably react with the drug or the solvent and possibly change the characteristics of the resulting drug or drug formulation particles. A suitable inert gas includes, without limitation, nitrogen. The moving volume of gas circulates through the drying vessel until it reaches the exhaust, at which point the gas is exhausted. According to one exemplary method, the moving volume of gas is established in a cyclone collector. An exemplary system that makes use of a cyclone collector will be discussed in more detail below.

The present exemplary method also includes jetting the drug-containing solution into the moving volume of gas (step 230). In particular, the drug-containing solution is jetted at a desired droplet size. As introduced, the droplets may be jetted at a maximum size of less than about 1 micron. Any suitable device may be used to provide droplets of the desired size. Such devices may include devices commonly referred to as inkjet type devices. For example, suitable devices may include, without limitation, thermally, magnetically, and/or piezo-electrically actuated inkjet type devices.

As the drug enters the moving volume of gas, the solvent in the drug-containing solution is rapidly evaporated. The rapid evaporation of the solvent may reduce the possibility that the drug will form a crystalline structure. Further, such evaporation yields a rapidly quenched drug or drug formulation particle in a high-energy, metastable solid state. Further, the moving volume of gas may be a generally laminar gas flow that remains laminar as it is circulated past the jetting devices. The laminar flow of the gas may allow the droplets to form substantially spherical particles.

In particular, the substantially laminar flow frequently is less disruptive than a turbulent flow. Thus, a laminar flow is less likely to disrupt the shape of a small droplet. At such sizes, small droplets are frequently generally spherical. Accordingly, a generally laminar flow may allow the droplets to retain their generally spherical shape as they dry. As the droplets dry, their size decreases such that the final drug particles are substantially smaller than the original droplets. Once the solvent is evaporated, the resulting solidified drug or drug formulation particles may be substantially round and have an average size of less than about 1 micron.

One exemplary method also includes the monitoring of the size of the droplets and resulting particles (step 240). The monitoring of droplet and particle sizes may occur at several stages at several locations. For example, a first sensing operation may be used to measure the average size of the droplets, such as monitoring the location near where the droplets are injected into the moving gas flow, or by placing the sensing device at the area of droplet injection. Such a measurement may be taken using visual sensors or other sensing operations commonly known in the art. Further, subsequent sensing operations may be used to ensure the droplet sizes are within a desired size range.

In particular, it is determined whether the measured values of the droplets, and thus the resulting particles, are below a predetermined threshold (determination 245). According to one exemplary embodiment, the predetermined threshold is about 1 micron. Such a range may correspond to a desired size of a droplet upon being injected into the gas flow and a desired final size of the particles. If the droplets are within the predetermined range (YES, determination 245), the operation continues as normal. If, however, it is determined that the droplets are larger than the desired range, (NO, determination 245), the controller adjusts the process conditions (step 248), such as the jetting operation and/or the properties of the cyclone collector discussed above.

For example, sensors may be used to monitor the size of the jetted and drying droplets and adjust the jetting conditions of the jetting devices and/or the flow and/or temperature conditions of the moving gas volume to help ensure proper particle size. In particular, an upper sensor may monitor the size of the droplets shortly after they are injected into the moving gas volume. Suitable sensors include, without limitation, droplet size analyzers or other optical sensors, as are known in the art. The sensors transmit this information to the controller. The controller compares the sensed size of the droplets to a predetermined range of droplet sizes and to determine if average droplet size is increasing. If the controller determines the average droplet size is above a predetermined maximum, the controller adjusts the injection of the droplets to bring the droplet to sizes within the predetermined range. For example, the controller may adjust dispensing rate, resistor energies such as voltage, pulse width, and pulse warming or other factors to control the size of the droplets.

Lower sensors may monitor the average size of the drying droplets as they dry. If the controller determines that the average droplet size is increasing, the controller may determine that the moving volume of gas is too rich in solvent. For example, the moving volume of gas may efficiently dry the droplets to a target size, but a vapor content that is too high may result in aggregation of sticky droplets into larger droplet agglomerates, indicated by an increasing droplet size as they move down the conical portion to the collection vessel. If the controller determines such a condition, the controller adjusts the jetting rate and/or the flow rate and temperature to thereby reduce the concentration of evaporated solvent in the moving volume of gas.

Once the particles are processed, the particles are then collected (step 250) and the solvent rich gas is exhausted (step 260). The exhausted gas may be vented or introduced to a recovery system to capture solvent vapor for recycling or disposal. Accordingly, the present method provides for the jetting of a drug-containing solution into a moving volume of gas or a gas flow to form relatively small particles of a rapidly quenched drug or drug formulation particle in a high-energy, metastable solid state. In bulk, such particles may form a free-flowing powder. Such a powder may be readily processed and/or dissolved. One exemplary system of forming such particles will now be discussed in more detail.

Drug Processing System with Jetting Devices and a Cyclone Collector

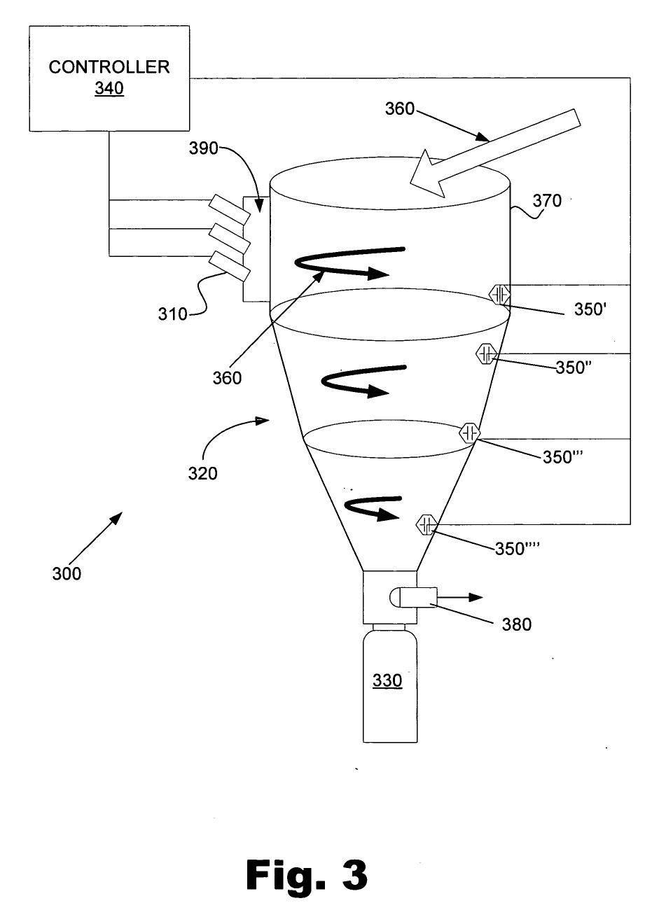

FIG. 3 illustrates a drug processing system (300) according to one exemplary embodiment. The drug processing system (300) generally includes jetting devices (310), a cyclone collector (320), a collection vessel (330), a controller (340), and sensors (350′, 350″, 350′″, 350″″).

According to the present exemplary embodiment, the controller (340) controls the operation of the cyclone collector (320). In particular, the controller (340) controls the volumetric flow rate and the temperature of the temperature of a gas flow (360) entering a conical portion (370) of the cyclone collector (320). The gas flow (360) circulates through the conical portion (370) and generally swirls around the walls of the conical portion (370) from the top of the conical portion (370) toward the bottom of the conical portion (370) to an exhaust (380). According to the present exemplary embodiment, the cyclone collector (320) causes the gas flow (360) to flow in a generally laminar manner as it circulates. As the gas flow (360) reaches the exhaust (380), the gas flow is exhausted from the cyclone collector (320).

The illustrated jetting devices (310) may be part of a thermal inkjet printhead, although other types of configurations may be used in conjunction with the present system and method, including, but in no way limited to, thermally actuated inkjet dispensers, mechanically actuated inkjet dispensers, electrostatically actuated inkjet dispensers, magnetically actuated dispensers, piezo-electrically actuated inkjet dispensers, continuous inkjet dispensers, etc.

Each of the jetting devices may include resistors which are associated with a nozzle. Further, each of the jetting devices (310) is in fluid communication with a supply of drug-containing liquid such that an amount of drug-containing liquid is present in each of the nozzles. The controller (340) is configured to selectively energize the resistors. Upon energizing a selected resistor a bubble of gas is formed which ejects a droplet of the drug-containing liquid from the nozzle.

According to one exemplary embodiment, the droplets have a maximum size of less than about 1 micron. These relatively small droplets are jetted into a space (390) formed between the jetting devices (310) and an opening in the cyclone collector (320). Jetting the droplets into the space (390) may reduce the amount of drug-containing fluid that is unintentionally drawn into the cyclone collector (320). The space (390) may also serve to increase the dispersal of the injected droplets before they enter the drying area (320). It may also be desirable to introduce a small, controlled flow of gas through the space (390) to optimize this dispersal of injected droplets.

For example, as fluid flows past a given point, the pressure at that point is decreased. The faster the flow, the more the pressure will be decreased. Thus, as the drying gas flows past the opening in the wall of the cyclone collector near the jetting devices (310), the pressure at an opening in the wall is reduced, thereby creating a pressure gradient between the gas flow and the opening in the wall.

The space (390) between the nozzles or outlets of the jetting devices (310) provides distance between the low pressure area and the nozzles, thereby reducing the pressure gradient between the gas flow and the jetting devices (310). The introduction of a small gas flow through the space (390) may also serve to decrease the pressure gradient. Reducing the pressure gradient may reduce the possibility that drug-containing fluid in the jetting devices will be unintentionally drawn into the gas flow. Rather, the drug-containing fluid is selectively jetted into the space (390).

As the droplets approach the low pressure zone created by the gas flow described above, the droplets are drawn into the gas flow (360) and thus are drawn into the cyclone collector (320). As introduced, the cyclone collector (320) causes the gas to flow in a generally laminar manner as it circulates. This generally laminar flow may reduce disruptions in the shape of the droplets as they enter the gas flow (360) and are dried therein. Reducing such disruptions may allow the droplets to form substantially spherical particles.

Further, as introduced, the gas flow (360) may cause the solvent of the drug-containing solution to evaporate rapidly. As the gas flow (360) carries the droplets around the conical portion (370), an increasing portion of the solvent evaporates, causing the resulting droplet to be smaller and smaller until a solid particle and solvent gases remain.

The sensors (350′, 350″, 350′″, 350″″) monitor the size of these droplets and resulting particles and adjust jetting conditions of the jetting devices (310) and/or the flow and/or temperature conditions of the gas flow (360) to help ensure proper particle size. In particular, the upper sensor (350′) may monitor the size of the droplets shortly after they are injected into the gas flow (360). The sensor (350′) transmits this information to the controller (340). The controller (340) compares the sensed size of the droplets to a predetermined range of droplet sizes. If the controller (340) determines the droplet size is outside the predetermined range, the controller (340) adjusts the injection of the droplets accordingly to bring the droplet to sizes within the predetermined range. In particular, the controller (340) may adjust dispensing rate, resistor energies such as voltage, pulse width, and pulse warming or other factors to control the size of the droplets.

The other sensors (350″, 350′″, 350″″) may be used to determine whether individual droplets are sticking together and/or determine information about the vapor environment in the gas flow (360). In particular, the other sensors (350″, 350′″, 350″″) may function as particle size analyzers located at sight-glass ports in the cyclone collector to provide additional feedback to the controller (340). For example, the gas flow (360) may efficiently dry the droplets to a target size, but a vapor content that is too high may result in aggregation of sticky droplets into larger particle agglomerates, indicated by an increasing droplet size as they move down the conical portion (370) to the collection vessel (330).

If the controller (340) determines that the sensors (350″, 350′″, 350″″) are detecting such an increase, the controller (340) may change conditions within the system to reduce the solvent to gas ratio in the gas flow. For example, the controller may cause the jetting devices (310) to decrease the jetting rate. Further, the controller (340) may also cause an increase in the gas flow rate to thereby reduce the solvent to gas ratio while either holding the jetting rate constant or by reducing the jetting rate. Such control may cause rapid and controlled evaporation of the droplets injected into the gas flow while reducing the possibility that the drying droplets will stick together.

Such rapid evaporation may reduce the formation of a crystalline structure in the resulting drug particle. As a result, the resulting particles may be substantially amorphous particles with an average size of less than about 1 micron. As introduced, the gas flow (360) swirls around the walls of the conical portion (370). When particles of drug are present in the gas flow (360), these portions have a continually decreasing diameter that results in increased gas velocity as the flow passes down the conical area. This increased gas velocity in a circular path yields increased centrifugal force that causes the particles to be forced against the walls. At some point, the centrifugal force is sufficient that the particles will be separated from the gas flow (360) and will be dropped into the collection vessel (330). Thereafter, the gas flow (360), which is now rich with evaporated solvent, is removed through the exhaust (380).

As discussed, the resulting particles collected in the collection vessel (330) constitute a free-flowing powder with an average particle size as small as about 1 micron or less. Such a particle size may increase the aqueous solubility of the drug as discussed above.

In conclusion, a method and system have been provided herein that make use of a material jetting device to produce solid drug particles of relatively small particle size by dispensing small droplets of drug solution into a moving body of gas. The rapid evaporation of droplets that occurs yields a rapidly quenched drug or drug formulation particle in a high-energy, metastable solid state. For example, the resulting solid may be an amorphous particle with a relatively small particle size. Any version of a material jetting device can be used.

According to one exemplary embodiment, the material jetting device includes nozzles that are in communication with a body of drying gas. In particular, according to such an exemplary embodiment, a series of nozzles dispenses laterally into a circular flow of drying gas in a cyclone collector that rapidly dries the droplets to yield solid particles. The circulating flow of gas moves down through a conical collector to separate the particles by centrifugal force into a collection vessel while the particle lean gas moves out to be exhausted. Dried particles of uniform size, such as a particle size of less than about 1 μm, and approximately spherical in shape are produced. This method provides for the continuous monitoring and control of the dispensing conditions and generation of uniformly sized drug particles.

The preceding description has been presented only to illustrate and describe the present method and apparatus. It is not intended to be exhaustive or to limit the disclosure to any precise form disclosed. Many modifications and variations are possible in light of the above teaching. It is intended that the scope of the disclosure be defined by the following claims.

What is claimed is:

1. A method, comprising:
dissolving a drug in a solvent to form a drug-containing solution; and
jetting droplets of said drug-containing solution into a moving volume of gas, said droplets having a maximum size of about 1 micron.

2. The method of claim 1, wherein jetting said droplets includes jetting said droplets with an inkjet type device.

3. The method of claim 2, wherein jetting said droplets includes jetting said droplets with at least one of a thermally actuated, a magnetically actuated, or piezo-electrically actuated inkjet.

4. The method of claim 1, wherein dissolving said drug in a solvent includes dissolving said drug in at least one of a low boiling point alcohol, ether, ketone, ester, or a halogenated solvent.

5. The method of claim 1, wherein jetting said droplets of said drug-containing solution includes jetting said droplets into an inert gas.

6. The method of claim 1, wherein said inert gas includes nitrogen.

7. The method of claim 1, further comprising monitoring an average size of said droplets and reducing a jetting rate of said droplets when said average size of said droplets exceed a maximum average size.

8. The method of claim 1, and further comprising drying said droplets to form drug-containing particles.

9. The method of claim 8, further comprising monitoring an average size of said droplets as said droplets dry, determining whether said droplets are increasing in size, and reducing a solvent concentration in said moving volume of gas when said average size of said droplets increases.

10. The method of claim 1, and further comprising reducing a pressure gradient between a device used in jetting said droplets and said moving volume of gas.

11. The method of claim 1, wherein reducing said pressure gradient includes jetting said drug-containing solution into a space adjacent said moving volume of gas.

12. The method of claim 1, wherein jetting said droplets into said moving volume of gas includes jetting said droplets into a cyclone collector.

13. The method of claim 1, wherein jetting said droplets into said moving volume of gas includes jetting said droplets into a laminar gas flow.

14. A system for processing drugs, comprising:
a drying vessel configured to have a gas flow established therein; and
a jetting device in liquid communication with said drying vessel, said jetting device being configured to selectively jet droplets of drug-containing solution into said drying vessel at a maximum size of less than about 1 micron.

15. The system of claim 14, wherein said jetting device includes at least one of a thermally actuated, a magnetically activated, or a piezo-electrically actuated inkjet type device.

16. The system of claim 14, wherein said drying vessel comprises a cyclone collector.

17. The system of claim 14, further comprising at least one sensor and a controller, said controller being coupled to said sensor, said jetting device, and said drying vessel.

18. The system of claim 17, wherein said controller is configured to control said jetting device to produce smaller droplets in response to a detection by said sensor of a presence of a droplet larger than said maximum size.

19. The system of claim 17, wherein said controller is configured to control at least one of said jetting device and drying vessel to reduce a solvent to gas ratio in said drying vessel.

20. The system of claim 17, further comprising a space defined between said jetting device and said drying vessel, said space being configured to have said droplets jetted therethrough into said drying vessel.

21. A system, comprising:
means for generating a moving gas volume; and
means for jetting droplets of drug-containing solution into said moving gas volume to form drug particles of less than about 1 micron.

22. The system of claim 21, further comprising means for separating said drug particles from said moving gas volume.

23. The system of claim 22, further comprising means for sensing a size of said particles.

24. The system of claim 23, further comprising means for reducing a solvent to gas ratio in response to an input from said means for sensing a size of said particles.
